(19) United States
(12) Patent Application Publication
(10) Pub. No.: US 2004/0250730 A1
(43) Pub. Date: Dec. 16, 2004

(54) Calcium phosphate cements prepared from silicate-phosphate solutions

(76) Inventors: David Delaney, Scotts Valley, CA (US); Brent R. Constantz, Cupertino, CA (US)

Correspondence Address: BOZICEVIC, FIELD & FRANCIS LLP, 1900 UNIVERSITY AVE, SUITE 200, EAST PALO ALTO, CA, 94303, US

(21) Appl. No.: 10/462,075

(22) Filed: Jun. 12, 2003

(51) Int. Cl. A61F 2/28

(52) U.S. Cl. 106/35; 623/23.62; 106/690; 106/691

(57) ABSTRACT

Methods are provide for producing flowable compositions, e.g. pastes, that set into calcium phosphate products. In the subject methods, dry reactants that include a calcium source and a phosphate source are combined with a silicate-phosphate setting fluid, and the combined liquids and solids are mixed to produce the flowable composition. Also provided are the compositions themselves as well as kits for preparing the same. The subject methods and compositions produced thereby find use in a variety of applications, including the repair of hard tissue defects, e.g., bone defects.

### INTRODUCTION

1. Field of the Invention

The field of this invention is calcium phosphate cements.

2. Background

Calcium phosphate cements that are prepared by combining a dry component(s) and a liquid to form a flowable paste-like material that is subsequently capable of setting into a solid calcium phosphate product hold great promise for use as structural materials in the orthopedic and dental fields. For example, it is desirable to be able to inject a flowable material into a cancellous bone void and have the material set into a solid calcium phosphate mineral product that is capable of withstanding physiological loads. Materials that set into solid calcium phosphate mineral products are of particular interest as such products can closely resemble the mineral phase of natural bone and are susceptible to remodeling, making such products extremely attractive for use in orthopedics and related fields.

While a large number of different calcium phosphate cement formulations have been developed, there is a continued need for the development of yet more advanced formulations. Of particular interest is the development of formulations that set in a clinically relevant period of time into products that have sufficient strength to serve as cancellous and cortical bone substitutes and are capable of being replaced over time with natural bone.

3. Relevant Literature

Patents of interest include: U.S. Pat. Nos. 6,375,935; 6,139,578; 6,027,742; 6,005,162; 5,997,624; 5,976,234; 5,968,253; 5,962,028; 5,954,867; 5,900,254; 5,697,981; 5,695,729; 5,679,294; 5,580,623; 5,545,254; 5,525,148; 5,281,265; 4,990,163; 4,497,075; 4,429,691; 4,161,511 and 4,160,012.

### SUMMARY OF THE INVENTION

Methods are provided for producing settable compositions, e.g., injectable pastes and other flowable compositions, that set into calcium phosphate products. In the subject methods, dry reactants that include a calcium source and a phosphate source are combined with a silicate-phosphate setting liquid, e.g., a solution of a soluble silicate, such as sodium silicate, and phosphate. The combined liquids and solids are mixed to produce the settable composition. Also provided are the compositions themselves as well as kits for preparing the same. The subject methods and compositions produced thereby find use in a variety of applications, including the repair of hard tissue defects, e.g., bone defects.

### DESCRIPTION OF THE SPECIFIC EMBODIMENTS

Methods are provided for producing settable compositions, e.g., injectable pastes, which set into calcium phosphate products. In the subject methods, dry reactants that include a calcium source and a phosphate source are combined with a silicate-phosphate setting liquid, e.g., a solution of a soluble silicate (such as sodium silicate) and a phosphate. The combined liquids and solids are then mixed to produce the settable composition. Also provided are the compositions themselves as well as kits for preparing the same. The subject methods and compositions produced thereby find use in a variety of applications, including the repair of hard tissue defects, e.g., bone defects.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

It must be noted that, as used in this specification and the appended claims, the singular forms “a,” “an” and “the” include plural reference unless the context clearly dictates otherwise. Unless defined otherwise all technical and scientific terms used herein have the same meaning as commonly understood to one of ordinary skill in the art to which this invention belongs.

Where a range of values is provided, it is understood that each intervening value, to the tenth of the unit of the lower limit, unless the context clearly dictates otherwise, between the upper and lower limit of that range and any other stated or intervening value in that stated range, is encompassed within the invention. The upper and lower limits of these smaller ranges may independently be included in the smaller ranges, and such embodiments are also encompassed within the invention, subject to any specifically excluded limit in the stated range. Where the stated range includes one or both of the limits, ranges excluding either or both of those included limits are also included in the invention.

All publications mentioned herein are incorporated herein by reference for the purpose of describing and disclosing components that are described in the publications that might be used in connection with the presently described invention.

In further describing the subject invention, the subject methods will be described first, followed by a description of the compositions produced thereby, kits for use in preparing the same and methods for using the subject compositions in methods of hard tissue, e.g. bone repair.

### METHODS

In the subject methods, dry reactants that include a calcium source and a phosphate source are combined with a silicate-phosphate setting fluid under conditions sufficient to produce a settable, e.g., flowable, composition that sets into a calcium phosphate containing product, even when immersed in a fluid environment.

A feature of the subject methods is that a silicate-phosphate setting fluid is employed. By silicate-phosphate setting fluid is meant a fluid, e.g., solution, that includes both silicate and phosphate ions. In other words, it is a solution that includes both soluble phosphate and soluble silicate. As such, it is an aqueous solution of both phosphate and silicate ions, in which a silicate compound is dissolved and/or suspended and a phosphate compound is dissolved and/or suspended.

The silicate compound may be any compound that is physiologically compatible and is soluble in water. By soluble in water is meant a concentration of at least about 1%, usually at least about 2% and more usually at least about 5%, where the concentration of the silicate employed typically ranges from about 0-0.1 to 20%, including from about 0.01-5 to 15% and more usually from about 5 to 10%.

Representative silicates of interest include, but are not limited to: sodium silicates, potassium silicates, borosilicates, magnesium silicates, aluminum silicates, zirconium silicates, potassium aluminum silicates, magnesium aluminum silicates, sodium aluminum silicates, sodium methylsilicates, potassium methylsilicates, sodium butylsilicates, sodium propylsilicates, lithium propylsilicates, triethanol ammonium silicates, tetramethanolamine silicates, zinc hexafluorosilicate, ammonium hexafluorosilicate, cobalt hexafluorosilicate, iron hexafluorosilicate, potassium hexafluorosilicate, nickel hexafluorosilicate, barium hexafluorosilicate, hydroxyammonium hexafluorosilicate, sodium hexafluorosilicate and calcium fluorosilicate. The preparation of sodium hexafluorosilicate is described in U.S. Pat. Nos. 4,161,511 and 4,160,012; the disclosures of which are herein incorporated by reference. Of particular interest in many embodiments are solutions of sodium silicate, where the manufacture of dry sodium silicate (Na₂SiO₃, Na₆Si₂O₇ and Na₂Si₃O₇) is described in Faith, Keyes & Clark's INDUSTRIAL CHEMICALS (1975) pp 755-761. Also of interest is Na₄SiO₄ (CAS #1344-09-8).

The concentration of phosphate ion in the setting fluid may vary, but is typically at least about 0.01 mol/L, such as at least about 0.02 mol/L and more including at least about 0.025 mol/L, where the concentration may range from about 0.01 to about 0.5, such as from about 0.01 to about 0.25, including from about 0.02 to about 0.2 mol/L. The desired phosphate concentration may be provided using any convenient phosphate source, such as a non-calcium-containing salt of phosphoric acid that is sufficiently soluble, e.g., Na₃ PO₄, Na₂ HPO₄, or NaH₂ PO₄. Salts of other cations such as K⁺, NH₄⁺, etc., may also be employed.

In the subject methods, the above-described silicate-phosphate setting fluid is combined with dry reactants that include a calcium source and a phosphate source under conditions sufficient to produce a settable, e.g., flowable composition. The dry reactants that are combined with the setting fluid are typically particulate compositions, e.g. powders, where the particle size of the components of the particulate compositions typically ranges from about 1 to 1000 microns, usually from about 1 to 200 um and more usually from about 1 to 40 microns.

As mentioned above, the dry reactants include a calcium source and a phosphate source. The calcium source and phosphate source may be present as a single compound or present as two or more compounds. As such, a single calcium phosphate present in the dry reactants may be the calcium source and the phosphate source. Alternatively, two or more compounds may be present in the dry reactants, where the compounds may be compounds that include calcium, phosphate or calcium and phosphate. Calcium phosphate sources of interest that may be present in the dry reactants include: MCPM (monocalcium phosphate monohydrate or Ca(H₂PO₄)₂.H₂O); DCPD (dicalcium phosphate dihydrate, brushite or CaHPO₄.2H₂O), ACP (amorphous calcium phosphate or Ca₃(PO₄)₂H₂O), DCP (dicalcium phosphate, monetite or CaHPO₄), tricalcium phosphate, including both α- and β-(Ca₃(PO₄)₂, tetracalcium phosphate (Ca₄(PO₄)₂O, etc. Calcium sources of interest include: calcium carbonate (CaCO₃), calcium oxide (CaO), calcium hydroxide (Ca(OH)₂) and the like. Phosphate sources of interest include: Phosphoric acid (H₃PO₄), all soluble phosphates, and the like.

The ratios or relative amounts of each of the disparate calcium and/or phosphate compounds in the dry reactant mixture is one that provides for the desired calcium phosphate product upon combination with the silicate-phosphate setting fluid and subsequent setting. In many embodiments, the overall ratio (i.e., of all of the disparate calcium and/or phosphate compounds in the dry reactants) of calcium to phosphate in the dry reactants ranges from about 4:1 to 0.5:1, usually from about 2:1 to 1:1 and more usually from about 1.9:1 to 1.33:1.

A variety of calcium phosphate cement compositions are known to those of skill in the art, and such cements may be readily modified into cements of the subject invention by substituting a silicate containing solution for the setting solution of those cements. Cement compositions known to those of skill in the art and of interest include, but are not limited to, those described in U.S. Pat. Nos. 6,027,742; 6,005,162; 5,997,624; 5,976,234; 5,968,253; 5,962,028; 5,954,867; 5,900,254; 5,697,981; 5,695,729; 5,679,294; 5,580,623; 5,545,254; 5,525,148; 5,281,265; 4,990,163; 4,497,075; and 4,429,691; the disclosures of which are herein incorporated by reference.

One or both of the above liquid and dry reactant components may include an active agent that modulates the properties of the product into which the flowable composition prepared by the subject method sets. Such additional ingredients or agents include, but are not limited to: organic polymers, e.g., proteins, including bone associated proteins which impart a number of properties, such as enhancing resorption, angiogenesis, cell entry and proliferation, mineralization, bone formation, growth of osteoclasts and/or osteoblasts, and the like, where specific proteins of interest include, but are not limited to: osteonectin, bone sialoproteins (Bsp), α-2HS-glycoproteins, bone Gla-protein (Bgp), matrix Gla-protein, bone phosphoglycoprotein, bone phosphoprotein, bone proteoglycan, protolipids, bone morphogenic protein, cartilage induction factor, platelet derived growth factor, skeletal growth factor, and the like; particulate extenders; inorganic water soluble salts, e.g. NaCl, calcium sulfate; sugars, e.g. sucrose, fructose and glucose; glycosaminoglycans, GAGS, complex carbohydrates, gums (e.g., xanthan, hyaluronic acid); pharmaceutically active agents, e.g. antibiotics; and the like

In practicing the subject methods, suitable amounts of the dry reactants and the setting fluid are combined to produce a settable or flowable composition. In other words, the ratio of the dry reactants to setting fluid (i.e. the liquid to solids ratio) is selected to provide for a settable of flowable composition, where by settable or flowable composition is meant a composition that goes from a first non-solid (and also non-gaseous) state to a second, solid state after setting. In many embodiments, the liquid to solids ratio is chosen to provide for a flowable composition that has a viscosity ranging from that of milk to that of modeling clay. As such, the liquids to solids ratio employed in the subject methods typically ranges from about 0.2 to 1.0, usually from about 0.3 to 0.6. Of particular interest in many embodiments are methods that produce a paste composition, where the liquid to solids ratio employed in such methods typically ranges form about 0.25 to 0.5, usually from about 0.3 to 0.45.

Because the setting fluid is a silicate-phosphate setting fluid, certain features are realized that are not obtained with other types of setting fluids, where these features include: lubricity in combination with setting acceleration, etc.

As mentioned above, the requisite amounts of dry reactants and setting fluid are combined under conditions sufficient to produce the flowable product composition. As such, the dry and liquid components are typically combined under agitation or mixing conditions, such that a homogenous composition is produced from the dry and liquid components. Mixing may be accomplished using any convenient means, including manual mixing as described in U.S. Pat. No. 6,005,162 and automated mixing as described in WO 98/28068, the disclosures of which are herein incorporated by reference. Also of interest is the device disclosed in U.S. Pat. No. 5,980,482, the disclosure of which is herein incorporated by reference.

In certain embodiments, a simple cylindrical tube may be used both as a storage and packaging device and a mixing and delivery device. The plastic tube is separated into at least two sections, compartments or portions. One section or portion contains the powder component, as described above. The at least one more compartment contains the setting fluid, where in certain embodiments, two or more compartments for setting fluid components are provided, e.g., where it is desired to keep the disparate components of the setting fluid separate prior to use, and/or where one desires to have flexibility in determining the amounts of the phosphate and silicate ions in the setting fluid that is employed. For example, one may have a two-compartment device with powder in one component and a setting fluid in the other. In other embodiments, one may have a three compartment device, with powder in a first compartment, silicate solution in a second compartment and phosphate solution in a third compartment. In yet other embodiments, one may have a multi-compartment device, with powder in a first compartment, a solution at one concentration of either or both component ions in a second compartment, and a solution at a second concentration of either or both component ions in a third compartment, etc., where this type of embodiment allows one to “tailor” the setting fluid employed depending on the particular application in which the cement is to be used. In yet other embodiments, one may have a three-compartment device with powder in the middle component and setting solution in the two outer components, where each setting solution may be the same or different.

The two or more compartments are separated from each other by an easily removable barrier that can be readily removed during preparation of the packaged cement. Any convenient removable barrier may be present in the device, where a representative barrier means of interest is a dialysis bag clip or analogous means. Another representative barrier means of interest is a frangible barrier, as described in WO 98/28068 and U.S. Pat. No. 5,362,654; the disclosures of which are herein incorporated by reference. When one is ready to mix, the clip or other barrier means between the to areas (liquid(s) and powder) is removed (e.g., unclipped), and the contents are simply kneaded together by hand. The above steps may be performed through a second outer covering for sterility—i.e., the above-described package elements may be present in a second outer covering for sterility. The outer covering may then be removed and the mixed contents from the tube may be delivered from one end of the storage/mixing tube using a peristaltic action. This mixing device is exceedingly simple to use and inexpensive to supply, with no additional components necessary;—the entire mixing device is disposable. This device provides advantages over that described in U.S. Pat. No. 5,980,482.

The temperature of the environment in which combination or mixing of the dry and liquid components takes place is sufficient to provide for a product that has desired setting and strength characteristics, and typically ranges from about 0 to 50° C., usually from about 20 to 30° C. Mixing takes place for a period of time sufficient for the flowable composition to be produced, and generally takes place for a period of time ranging from about 15 to 100 seconds, usually from about 15 to 50 seconds and more usually from about 15 to 30 second.

The above described protocols result in a flowable composition that is capable of setting into a calcium phosphate mineral product, as described in greater detail below.

### SETTABLE/FLOWABLE COMPOSITIONS

The flowable compositions produced by the above-described methods are ones that set into a biologically compatible, and often resorbable and/or remodelable, product, where the product is characterized by including calcium phosphate molecules not present in the initial reactants, i.e., that are the product of a chemical reaction among the initial reactants. The term flowable is meant to include paste-like compositions, as well as more liquid compositions. As such, the viscosity time of the subject flowable compositions, defined as time periods under which the mixed composition injects through a standard Luer-lok fitting after mixing, typically ranges up to about 10 minutes, usually up to about 7 minutes, such as up to about 4 minutes. Of particular interest in many embodiments are paste compositions that have an injectable viscosity that injects in a time period ranging up to about 5 minutes, such as about up to about 4 minutes. Pastes that stay paste-like for longer period may be displaced by bleeding bone once implanted into the body, which create a blood interface between the cement and the bone prior to the cement hardening.

The compositions produced by the subject invention set into calcium phosphate mineral containing products. By calcium phosphate mineral containing product is meant a solid product that includes one or more, usually primarily one, calcium phosphate mineral. In many embodiments, the calcium phosphate mineral is one that is generally poorly crystalline, so as to be resorbable and, often, remodelable, over time when implanted into a physiologically site. The calcium to phosphate ratio in the product may vary depending on particular reactants and amounts thereof employed to produce it, but typically ranges from about 2:1 to 1.33:1, usually from about 1.8:1 to 1.5:1 and more usually from about 1:7:1 to 1.6:1. Of particular interest in many embodiments are apatitic products, which apatitic products have a calcium to phosphate ratio ranging from about 2.0:1 to 1.33:1, including both hydroxyapatite and calcium deficient analogs thereof, including carbonate substituted hydroxyapatite (i.e. dahllite), etc. The subject paste-like composition is, in many embodiments, preferably one that is capable of setting into a hydroxyapatitic product, and more preferably into a carbonated hydroxyapatite, i.e. dahilite, having a carbonate substitution of from about 2 to about 10%, usually from about 2 to about 8% by weight of the final product.

The period of time required for the compositions to harden or “set” may vary. By set is meant: the Gilmore Needle Test (ASTM C266-89), modified with the cement submerged under 37° C. physiological saline. The set times of the subject cements may range from about 30 second to 30 minutes, and will usually range from about 2 to 15 minutes and more usually from about 4 to 12 minutes. In many embodiments, the flowable composition sets in a clinically relevant period of time. By clinically relevant period of time is meant that the paste-like composition sets in less than about 20 minutes, usually less than about 15 minutes and often in less than about 10 minutes, where the composition remains flowable for at least about 1 minute, usually at least about 2 minutes and, in many embodiments, for at least about 5 minutes following combination or mixture of the precursor liquid and dry cement components. The use of phosphate-silicate setting fluids causes these cements to set faster than the same cements do when only water is employed; such that the rate of setting increases positively with increasing silicate concentrations.

The compressive strength of the product into which the flowable composition sets may vary significantly depending on the particular components employed to produce it. Of particular interest in many embodiments is a product that has a compressive strength sufficient for it to serve as at least a cancellous bone structural material. By cancellous bone structural material is meant a material that can be used as a cancellous bone substitute material as it is capable of withstanding the physiological compressive loads experienced by compressive bone under at least normal physiological conditions. As such, the subject flowable paste-like material is one that sets into a product having a compressive strength of at least about 10, including at least about 20, such as at least about 40 or at least about 50 MPa, as measured by the assay described in Morgan, E F et al., 1997, Mechanical Properties of Carbonated Apatite Bone Mineral Substitute: Strength, Fracture and Fatigue Behavior. J. Materials Science: Materials in Medicine. V. 8, pp 559-570., where the compressive strength of the final apatitic product may be as high as 60 MPa or higher. Inclusion of the silicate in the setting liquid allows lower liquid to solids ratios to be employed which results in significantly higher compressive strengths. Compressive strengths can be obtained that range as high 100 to 200 MPa. In certain embodiments, the resultant product has a tensile strength of at least about 0.5 MPa, such as at least about 1 MPa, including at least about 5 MPa, at least about 10 MPa or more, e.g., from about 0.5 to about 10 MPa, as determined by the tensile strength assay appearing in the Experimental Section, below.

In many embodiments, the resultant product is stable in vivo for extended periods of time, by which is meant that it does not dissolve or degrade (exclusive of the remodeling activity of osteoclasts) under in vivo conditions, e.g., when implanted into a living being, for extended periods of time. In these embodiments, the resultant product may be stable for at least about 4 months, at least about 6 months, at least about 1 year or longer, e.g., 2.5 years, 5 years, etc. In certain embodiments, the resultant product is stable in vitro when placed in an aqueous environment for extended periods of time, by which is meant that it does not dissolve or degrade in an aqueous environment, e.g., when immersed in water, for extended periods of time. In these embodiments, the resultant product may be stable for at least about 4 months, at least about 6 months, at least about 1 year or longer, e.g., 2.5 years, 5 years, etc.

As indicated above, the soluble phosphate and silicate act together to accelerate the setting reaction and provide lubricity for complete mixing of the cement reactants.

In certain embodiments, the flowable paste-like composition is capable of setting in a fluid environment, such as an in vivo environment at a bone repair site. As such, the flowable paste composition can set in a wet environment, e.g., one that is filled with blood and other physiological fluids. Therefore, the site to which the flowable composition is administered during use need not be maintained in a dry state.

In certain embodiments, the subject cement compositions may be seeded with any of a variety of cells. A “cell”, according to the present invention, is any preparation of living tissue, including primary tissue explants and preparations thereof, isolated cells, cells lines (including transformed cells), and host cells. Preferably, autologous cells are employed, but xenogeneic, allogeneic, or syngeneic cells are also useful. As such, the cells can be obtained directly from a mammalian donor, e.g., a patient's own cells, from a culture of cells from a donor, or from established cell culture lines. The mammal can be a mouse, rat, rabbit, guinea pig, hamster, cow, pig, horse, goat, sheep, dog, cat, and the mammal can be a human. Cells of the same species and preferably of the same immunological profile can be obtained by biopsy, either from the patient or a close relative. Where the cells are not autologous, it may be desirable to administer immunosuppressive agents in order to minimize rejection. In preferred embodiments, such agents may be included within the seeded composition to ensure effective local concentrations of the agents and to minimize systemic effects of their administration. The cells employed may be primary cells, explants, or cell lines, and may be dividing or non-dividing cells. Cells may be expanded ex-vivo prior to introduction into the inventive cement compositions. Autologous cells are preferably expanded in this way if a sufficient number of viable cells cannot be harvested from the host.

Any preparation of living cells may be use to seed the cement composition of the present invention. For example, cultured cells or isolated individual cells may be used. Alternatively or additionally, pieces of tissue, including tissue that has some internal structure, may be used. The cells may be primary tissue explants and preparations thereof, cell lines (including transformed cells), or host cells.

Any available methods may be employed to harvest, maintain, expand, and prepare cells for use in the present invention. Useful references that describe such procedures include, for example, Freshney, Culture of Animal Cells: a Manual of Basic Technique, Alan R. Liss Inc., New York, N.Y., incorporated herein by reference.

The cement composition material of the invention is useful as a scaffold for production of hard or soft tissues. Tissue-producing or -degrading cells that may be incorporated into the material include, but are not limited to, chondrocytes, osteocytes, osteoblasts, osteoclasts, mesenchymal stem cells, other bone- or cartilage-producing cells or cell lines, fibroblasts, muscle cells, hepatocytes, parenchymal cells, cells of intestinal origin, nerve cells, and skin cells.

Methods of isolating and culturing such tissue-producing or -degrading cells, and/or their precursors, are known in the art (see, for example, Vacanti et al., U.S. Pat. No. 5,041,138; Elgendy et al., Biomater. 14:263, 1993; Laurencin et al., J. Biomed. Res. 27:963, 1993; Freed et al., J. Cell. Biochem. 51:257, 1993; Atala et al., J. Urol. 150:745, 1993; Ishaug et al., J. Biomed. Mater. Res. 28:1445, 1994; Chu et al., J. Biomed. Mater. Res. 29:1147, 1995; Thomson et al., J. Biomater. Sci. Polymer Edn. 7:23, 1995, each of which is incorporated by reference).

For example, mesenchymal stem cells, which can differentiate into a variety of mesenchymal or connective tissues (including, for example, adipose, osseous, cartilagenous, elastic, and fibrous connective tissues), can be isolated, purified, and replicated according to known techniques (see Caplan et al., U.S. Pat. No. 5,486,359; Caplan et al., U.S. Pat. No. 5,226,914; Dennis et al., Cell Transplantation 1:23, 1992, each of which is incorporated herein by reference). Such mesenchymal cells have been studied in association with tricalcium phosphate and hydroxyapatite carriers and have been found to be capable of successful differentiation from within such carriers (see Caplan et al., U.S. Pat. No. 5,197,985, incorporated herein by reference). Similar procedures are employed to direct mesenchymal cell differentiation within the cement material of the present invention.

Of course, the present invention is not limited to the use of tissue-producing cells. Certain preferred embodiments of the invention utilize such cells, primarily because the inventive material is so well suited to tissue-regeneration applications (particularly with those involving growth of bone and/or cartilage). Any cell may be seeded into the material of the invention. In some cases, it will be desirable to include other cells in addition with tissue-producing cells.

Any convenient cell source may be employed. For example, where the material is seeded with stem cells, e.g., adult stem cells, mesenchymal stem cells, any convenient stem cell source may be employed. Stem cell sources of interest include bone marrow, cord blood, etc., which source may be treated to enrich the target stem cell population of interest, e.g., fractionated, etc.

The cells that are seeded into the inventive cement composition may be genetically engineered, for example to produce a protein or other factor that it useful in the particular application. In preferred embodiments, cells may be engineered to produce molecules that impart resistance to host immune attack and rejection. The Fas-L and CR-1 genes are examples of useful such genes.

Generally, cells are introduced into the subject material of the present invention in vitro, although in vivo seeding approaches are employed in some circumstances. Cells are typically mixed with the cement composition prior to setting.

Any available method may be employed to introduce the cells into the cement composition material. For example, cells may be injected into the flowable cement composition (sometimes in combination with growth medium), or maybe introduced by other means such as pressure, vacuum, or osmosis. Alternatively (or additionally), cells may be layered on the flowable cement composition. In certain embodiments, it may be desirable to manually mix or knead the cells with the material paste. Cells may also be introduced into the hydrated precursor in vivo simply by placing the material in the body adjacent a source of desired cells. In some cases, it may be desirable to enhance such in vivo cell impregnation by including within the material an appropriate chemotactic factor, associative factor (i.e., a factor to which cells bind), or factor that induces differentiation of cells into the desired cell type.

As those of ordinary skill will readily appreciate, the number of cells to be introduced into the inventive material will vary based on the intended application of the seeded material and on the type of cell used. Where dividing autologous cells are being introduced by injection into the hydrated precursor, use of 20,000-1,000,000 cells per cm³ are expected to result in cellular proliferation and extracellular matrix formation within the material. Where non-dividing cells are employed, larger numbers of cells will generally be required. In those cases where seeding is accomplished by host cell migration into the material in vivo, exposure of the material to fluids containing cells (e.g., bone-forming cells), or to tissue (e.g., bone) itself has proven to be effective to seed the material with cells without the need for inoculation with a specified number of cells. The use of cells as described above is further described in U.S. Pat. No. 6,139,578 and the references cited therein, the disclosures of which are herein incorporated by reference.

With respect to cell seeded cements, as described above, the subject cement is, in many embodiments, a structural cement. By structural cement is meant a cement composition that, upon setting, achieves a compressive strength of at least about 20, usually at least about 40 and more usually at least about 50 MPa, as measured by the assay described in Morgan, E F et al., 1997, Mechanical Properties of Carbonated Apatite Bone Mineral Substitute: Strength, Fracture and Fatigue Behavior. J. Materials Science: Materials in Medicine. V. 8, pp 559-570, where the compressive strength of the final apatitic product may be as high as 60 or higher, e.g., as high 100 to 200 MPa or higher. Cements other than the specific cements described herein that qualify as structural cements include, but are not limited to, those described in: U.S. Pat. Nos. 6,027,742; 6,005,162; 5,997,624; 5,976,234; 5,968,253; 5,962,028; 5,954,867; 5,900,254; 5,697,981; 5,695,729; 5,679,294; 5,580,623; 5,545,254; 5,525,148; 5,281,265; 4,990,163; 4,497,075; and 4,429,691; the disclosures of which are herein incorporated by reference.

Seeding a structural cement with pluripotent cells according to the above description results in stress induced cell differentiation of the pluripotent cells, e.g., into bone forming cells, i.e., osteoblasts. As such, the subject invention provides methods of differentiating pluripotent cells into bone forming cells via stress induction, wherein a sufficient amount of pluripotent cells are seeded in a structural cement as described above, which is subsequently allowed to set and, upon setting, results in stress induced differentiation of cells seeded therein as a result of mechanical forces applied to the set cement composition.

In the subject methods, it is important to employ a structural cement having a compressive strength that is comparable to cancellous bone, i.e., has a compressive strength of at least about 20 mPa. Such a feature is important because it is this feature that causes a stress induced differentiation of pluripotent cells present in the cement. As such, the term “structural cement” as used herein does not include cements that do not achieve a compressive strength of at least about 20 mPa, as such cements cannot provide the desired stress induced differentiation. Likewise, the term “structural cement” does not include a composition that sets into a structure which fractures in response to stress—,.e., the composition must remain as one homogeneous mass under applied physiological stresses and not be a composition that will fracture, as the latter type of composition gives rise to the formation of fibrous tissue, which is not a desirable outcome for the subject invention.

In addition, in certain embodiments the compositions include demineralized bone matrix, which may be obtained typically in lyophilized or gel form and is combined with the cement composition at some prior to implantation. A variety of demineralized bone matrixes are known to those of skill in the art and any convenient/suitable matrix composition may be employed.

### SPECIFIC REPRESENTATIVE CEMENT FORMULATIONS

A number of specific representative cement formulations have been developed and are of interest. These formulations include the following:

α-Tricalcium Phosphate and Monetite Formulations

One specific representative cement formulation of interest is an α-tricalcium phosphate and monetite cement formulation in which the dry reactant is a mixture (powder) of both α-tricalcium phosphate and monetite, where the mass ratio of tricalcium phosphate to monetite ranges from about 50/50 to about 90/20 in certain embodiments.

In this embodiment, the silicate-phosphate setting fluid is typically a fluid having a pH of from about 9 to 12, e.g., 10 to 12, such as about 11.5, with a silicate ion concentration of from about 0.02 to about 0.3M, and a phosphate concentration of from about 0.01 M to about 0.2 M.

α-Tricalcium Phosphate/Monetite/Hydroxyapatite Seed Formulations

Another specific representative cement formulation of interest is an α-tricalcium phosphate/monetite/hydroxyapatite seed cement formulation in which the dry reactant is a mixture (powder) of: α-tricalcium phosphate, monetite and hydroxyapatite seed, where the mass ratio of tricalcium phosphate to monetite ranges from about 50/50 to about 80/20.

In this embodiment, the silicate-phosphate setting fluid is typically a fluid having a pH of from about 9 to 12, e.g., 10 to 12, such as about 11.5, with a silicate ion concentration of from about 0.02 to about 0.3M, and a phosphate concentration of from about 0.01 M to about 0.2 M.

α-Tricalcium Phosphate/β-Tricalcium Phosphate/Monetite/Hydroxyapatite Seed Formulations

Yet another specific representative cement formulation of interest is an α- and β-tricalcium phosphate/monetite/hydroxyapatite seed cement formulation in which the dry reactant is a mixture (powder) of: α-tricalcium phosphate, β-tricalcium phosphate/monetite and hydroxyapatite seed, where the mass ratio of α-tricalcium phosphate to β-tricalcium phosphate ranges from about 90/10 to about 10/90, and the mass ratio of both tricalcium phosphates to monetite ranges from about 80/20 to about 50/50; and the hydroxyapatite seed is present in an amount from about 1 to about 20%.

In this embodiment, the silicate-phosphate setting fluid is typically a fluid having a pH of from about 9 to 12, e.g., 10 to 12, such as about 11.5, with a silicate ion concentration of from about 0.02 to about 0.3M, and a phosphate concentration of from about 0.01 M to about 0.2 M.

### APPLICATIONS

The subject methods and compositions produced thereby, as described above, find use in applications where it is desired to introduce a flowable material capable of setting up into a solid calcium phosphate product into a physiological site of interest, such as in dental, craniomaxillofacial and orthopedic applications. In orthopedic applications, the cement will generally be prepared, as described above, and introduced to a bone repair site, such as a bone site comprising cancellous and/or cortical bone. Orthopedic applications in which the cements prepared by the subject system find particular use include the treatment of fractures and/or implant augmentation, in mammalian hosts, particularly humans. In such fracture treatment methodologies, the fracture is first reduced. Following fracture reduction, a flowable structural material prepared by the subject system is introduced into the cancellous tissue in the fracture region using the delivery device described above. Specific dental, craniomaxillofacial and orthopedic indications in which the subject invention finds use include, but are not limited to, those described in U.S. Pat. No. 6,149,655, the disclosure of which is herein incorporated by reference. In addition to these particular applications described in this U.S. patent, the subject cement compositions also find use in applications where a sternotomy has been performed. Specifically, the subject cements find use in the closure process of a sternotomy, where the bone fragments are rejoined and wired together, and any remaining cracks are filled with the subject cement. In yet other embodiments, the subject compositions find use in drug delivery, where they are capable of acting as long lasting drug depots following administration to a physiological site. See e.g. U.S. Pat. Nos. 5,904,718 and 5,968,253; the disclosures of which are herein incorporated by reference.

### KITS

Also provided are kits comprising the subject cements, where the dry and liquid components may be present in separate containers in the kit, or some of the components may be combined into one container, such as a kit wherein the dry components are present in a first container and the liquid components are present in a second container, where the containers may or may not be present in a combined configuration, as described in U.S. Pat. No. 6,149,655, the disclosure of which is herein incorporated by reference. In certain embodiments, the kits may include two or more setting fluids in different concentrations, e.g., where one wishes to provide a kit with flexibility with respect to the nature of the setting fluid that is prepared therefrom. For example, a kit may include two more different phosphate-silicate solutions that differ from each other with respect to their silicate and/or phosphate components. Alternatively, the kit may include to or more different, separate phosphate and/or silicate solutions that differ from each other in terms of concentration and that are mixed upon use of the kit as desired to obtain a desired setting fluid. As mentioned above, the kit components may be present in separate containers. Alternatively, the components may be present as a packaged element, such as those described above.

In addition to the cement compositions, the subject kits may further include a number of additional reagents, e.g., cells (as described above, where the composition is to be seeded), protein reagents (as described above), and the like.

Kits can also include device for aspirating marrow, e.g., needle and trocar, which finds use for harvesting marrow (i.e. a pluripotent cell source) from a patient. Such components find use in those embodiments where the kit is employed in a method where the structure cement is seeded with the patient's own marrow, such as that described in Example 4, below.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

The following examples are offered by way of illustration and not by way of limitation.

### EXPERIMENTAL

### Example I

### A. Protocol Determination of Setting Time

1. Materials

Calcium Phosphate Cement to be tested

Phosphate buffered saline (Sigma Chemical Co, St. Louis)

Constant Temperature Water Bath (VWR, South San Francisco, Calif.)

Mechanical Testing Machine (Instron, Canton, Mass.)

Syringe (5 cc polypropylene. Terumo, Tokyo Japan.)

12 gauge, 10 cm needle (Henry Schein, Sussex, N.Y.)

Working time test containers (12 mm in diameter and 7 mm in length)

Modified high load indentor (7 mm in diameter)

2. Methodology

This test is a modification of the standard setting tests described in ASTM C403/C403M-99 in which the load required to drive needles a prescribed distance into concrete or similar setting materials is measured. This modified test employs a needle with a tip configuration similar to that used in ASTM C266-99. The needle is pushed 0.125 mm into the sample cured under physiologic conditions (immersed in PBS at 37° C.). An indentation load of greater than 9 Newton (2 lbs) is indicative of the initiation of set. The first evidence of measurable load above 9 Newton is used as the criteria in determining initiation of set, and interpolation may or may not be used as recommended in ASTM C403/C403M-99. This load is less than the load specified in the ASTM C403/C403M-99 setting test as the initiation of set test is a sensitive measure of the beginning of solidification of the cement, and serves to define the end of the work period at which time manipulation of the cement becomes difficult due to material hardening.

3. Procedure

The working time of the cement at 37° C. is determined as follows:

Testing is performed at 37° C. with a mechanical testing machine using a modified high-load indentor with the crosshead travel speed set to 15.2 mm/min and a maximum load of 5000 N. No spring load average was calculated or used since the high load indentor test fixture does not use a spring. The cement is prepared and backfilled into a 5 cc luer lock syringe with a 12 gauge, 10 cm needle. Working time containers are filled with the cement and, immediately after mixing, placed in PBS maintained at 37° C. One sample is removed from the PBS at each test time for testing. The indentor is positioned to be barely in contact with the sample but not to exceed a preload of 2.7 N (0.6 lbs) before initiating each test. Test times for each mix are chosen between 1, 3, 5, and 7 minutes postmix. After each sample is preloaded, the high load indentor is allowed to travel 0.125 cm into each sample and the final load and test time are recorded. The indentation load vs. time may be plotted and an exponential curve fit on the plot generated. The time at which the first evidence of indentation load (>9 Newton) is measured for samples cured at 37° C. is used to indicate the initiation of set.

### B. Setting Times for Representative Cements

1. Formulation 1

A 50:1 dilution of Sodium Silicate (Fisher) was further diluted in half by addition of 0.4M Na₂HPO₄. (i.e. 10 mls of 40:1 Sodium silicate added to 10 mls of 0.4M Na₂HPO₄). The final pH of the solution was=11.0. 1.65 g of the resulting solution was added to the powder component consisting of 2.6 g α-TCP, 1.0 g DCPA, and 0.4 g HA seed. Powder and solution components were mixed to form a cohesive paste in a glass mortar and pestle. The setting time for the resultant cement was measured according to Example 1 above.

Results for set testing were approximately 3 MPa after 20 min curing at 37° C.

2. Formulation 2

A 20:1 dilution of Sodium Silicate was further diluted (i.e. 17 ml 20:1 sodium silicate added to 10 ml 0.4M Na₂HPO₄). 0.75 g of the resulting solution was added to the powder components consisting of 1.3 g αTCP, 0.5 g DCPA, and 0.1 g HA seed. Powders and solution were mixed to form a cohesive paste in a glass mortar and pestle. The setting time for the resultant cement was measured according to Example 1 above. Results for set testing were approx. 3 MPa after 20 min curing at 37° C.

### Example II

### A. Additional Formulation 1

2 ccs of marrow are aspirated from an intermedullary access site, e.g., from an open fracture or percutaneously, from a patient. Cement prepared according to Example 2 is combined with harvested marrow by carefully folding the harvested marrow into the prepared paste cement composition without grinding, e.g., with a spatula. The resultant seeded mixture is then implanted into the bony defect site of the patient and allowed to set.

### B. Additional Formulation 2

Cement prepared according to Example 2 is combined with lyophilized demineralized bone matrix by carefully folding the matrix into the prepared paste cement composition without grinding, e.g., with a spatula. The resultant mixture is then implanted into the bony defect site of a patient and allowed to set.

### Example III

Objective: The purpose of following experiment was to determine the tensile strength of a bone cement set with a silicate setting fluid using a diametral tensile test.

Materials:

Callos™ Bone Void Filler (Skeletal Kinetics, Cupertino, Calif.)

MCPM: Calcium Phosphate, Monobasic, Monohydrate, Crystal (J T Baker Part#1426-01 Lot#T27597, Phillipsburg, N.J.)

DCPA: Calcium Phosphate Dibasic Anhydrous Powder (J T Baker Part#1430-07 Lot#25613, Phillipsburg, N.J.)

Sodium silicate solution (Fisher Part#SS338-1 Lot#9921928, Fair Lawn, N.J.)

Mechanical testing machine (Instron System ID 3366P6417, Canton, Mass.)

Analytical balance (Sartorius AG BP301 S Part#70802796, Goettingen, Germany)

PBS: 10× Phosphate buffered saline liquid concentrate (Sigma Chemical Co. OmniPur Part#6505 Lot#2933871, St.Louis, Ill.)

DI Reagent Grade Water (NERL Lot#1206211, East Providence, R.I.)

Constant temperature water bath (VWR Model#1210 Serial#0503788, South San Francisco, Calif.)

Mortar and pestle, polyethylene (NUNC, South San Francisco, Calif.)

Tensile testing cells and dividers (custom)

Procedure:

Diametral compression testing was performed on Callos™ disk shaped samples of 0.6 in. diameter and 0.3 in. thickness. Callos™ was mixed according to the package insert, using a mortar and pestle. The specimens were prepared by injecting Callos™ paste into small disk shaped capsules positioned on top of a square divider. To remove voids from the cement, the rings were vortexed. The rings were topped off with Callos™ paste. This process of filling sample rings was repeated three times for a total of three samples per 5 cc Callos™ reactants pack. Three more samples were generated with this process for a total of six samples. The filled sample rings were placed into a temperature controlled PBS bath maintained at 37° C. and allowed to cure for 24 hours. After 24 hours, sample rings were disassembled by carefully removing the caps, and the samples were pushed out of the ring.

Tensile strength testing was conducted using a screw-type Instron system. The Instron was connected to a PC operating MS Windows XP and data acquisition software. The Callos™ specimen was placed between compression platens and loaded to failure.

Samples were loaded at displacement rate of 0.1 in./min. Load, displacement, and time was recorded continuously at a sampling rate of 100 Hz. Ultimate tensile stress was calculated using the following equation:

Equation of tensile stress: σ=2P/πDt,

where:

P=ultimate compressive load, Newtons

D=sample diameter, millimeters

t=sample thickness, millimeters.

Results:

The mean tensile strength measured for Callos™ samples was 3.0±0.4 MPa. The results for this testing are found in Table 1.

TABLE 1
Summary table of tensile strength for each sample.

| Sample # | Strength (MPa) |
|---|---|
| 1 | 2.45 |
| 2 | 2.90 |
| 3 | 3.10 |
| 4 | 3.49 |
| 5 | 3.28 |
| Mean ± standard | 3.0 ± 0.4 MPa |
| deviation | |

Conclusions:

The tensile strength of cancellous bone has been reported to be in a wide range from 3 to 20 MPa (Tencer and K. D. Johnson. 1994. Biomechanics in Orthopedic Trauma, Bone Fracture and Fixation. In edited by Philadelphia: J. B. Lippincott Company). Based on these data, Callos™ exhibits tensile strength observed for the lower range reported for cancellous bone.

It is evident from the above results and discussion that calcium phosphate cements employing phosphate-silicate liquids may be mixed very quickly and easily without specialized mixing devices, set rapidly, and are able to obtain higher strengths due to the lower liquids to solids ratios employed. As such, the subject invention represents a significant contribution to the art.

All publications and patent applications mentioned in this specification are herein incorporated by reference to the same extent as if each individual publication or patent application was specifically and individually indicated to be incorporated by reference.

The invention now being fully described, it will be apparent to one of skill in the art that many changes and modifications can be made thereto without departing from the spirit and scope of the appended claims.

What is claimed is:

1. A method of producing a flowable composition that sets into a calcium phosphate containing product, said method comprising:
combining:
(a) a phosphate-silicate setting fluid; and
(b) dry reactants comprising a calcium source and a phosphate source;
wherein said setting fluid and dry reactants are combined in a ratio sufficient to produce said flowable material.

2. The method according to claim 1, wherein said ratio ranges from about 0.2:1 to 0.7:1.

3. The method according to claim 2, wherein said flowable composition is a paste.

4. The method according to claim 1, wherein said setting fluid is a solution of a soluble silicate having a silicate concentration ranging from about 0.1% to 15%.

5. The method according to claim 1, wherein said setting fluid has a phosphate concentration ranging from about 0.01 to about 0.5 M.

6. The method according to claim 1, wherein said flowable composition sets into said calcium phosphate containing product in a period of time ranging from about 5 to 10 minutes.

7. The method according to claim 1, wherein said calcium phosphate containing product has a compressive strength ranging from about 10 to 100 MPa.

8. A method of producing a paste that sets into a calcium phosphate containing product, said method comprising:
(a) combining:
(i) dry reactants comprising a calcium source and a phosphate source; and
(ii) a setting fluid having a silicate concentration ranging from about 0.1% to 10% and a phosphate concentration ranging from about 0.01 to 0.5M, wherein said dry reactants and setting fluid are combined in a ratio sufficient to provide for said paste; and
(b) mixing said combined reactants and setting fluid for a sufficient period of time to produce a paste capable of setting into a calcium phosphate containing product.

9. The method according to claim 8, wherein said ratio ranges from about 0.3 to 0.5.

10. The method according to claim 8, wherein said paste sets in a period of time ranging from about 4 to 10 minutes into a calcium phosphate containing product having a compressive strength ranging from about 40 to 70 MPa.

11. A flowable composition that sets into a calcium phosphate containing product, wherein said composition is produced by the method comprising:
combining dry reactants comprising a source of calcium and phosphate with a silicate-phosphate setting fluid; and
mixing said combined dry reactants setting fluid for a period of time sufficient to produce said flowable product.

12. The composition according to claim 11, wherein said composition is a paste.

13. The composition according to claim 11, wherein said calcium phosphate containing product is an apatitic product.

14. The composition according to claim 11, wherein said composition sets in period of time ranging from about 4 to 12 minutes.

15. The composition according to claim 11, wherein said calcium phosphate containing product has a compressive strength ranging from about 25 to 100.

16. A method of repairing a hard tissue defect, said method comprising:
applying to the site of said defect a flowable composition that sets into a calcium phosphate containing product, wherein said composition is produced by the method comprising:
(a) combining dry reactants comprising a source of calcium and phosphate with a silicate-phosphate setting fluid; and
(b) mixing said combined dry reactants and setting fluid for a period of time sufficient to produce said flowable product.

17. The method according to claim 16, wherein said composition is a paste.

18. A kit for use in a preparing a flowable composition that sets in an in vivo fluid environment into a calcium phosphate product, said kit comprising:
(a) dry reactants comprising a calcium source and a phosphate source; and
(b) a phosphate-silicate setting fluid or components for producing the same.

19. A packaged calcium phosphate cement, said packaged cement comprising:
(a) a tubular element separated into a first compartment and at least one additional compartment by a removable barrier;
(i) dry reactants comprising a source of calcium and phosphate present in said first compartment; and
(ii) a silicate-phosphate setting fluid or components thereof present in said at least one additional compartment.

20. The packaged calcium phosphate cement according to claim 19, wherein said removable barrier is a clip.

21. The packaged calcium phosphate cement according to claim 20, wherein said removable barrier is a frangible barrier.

22. The packaged calcium phosphate cement according to claim 19, wherein said tubular element is present inside a second sterility maintaining element.
